(19) United States
(12) Patent Application Publication
(10) Pub. No.: US 2025/0378928 A1
(43) Pub. Date: Dec. 11, 2025

(54) Method for Ventricular Activation Assessment from Regular Electrocardiogram Using Neural Network

(71) Applicant: VDI Technologies s.r.o., Brno (CZ)

(72) Inventors: Filip Plesinger, Brno (CZ); Zuzana Koscova, Krompachy (SK); Pavel Jurak, Brno (CZ); Ivo Viscor, Brno (CZ); Radovan Smisek, Kurim (CZ); Miroslav Navratil, Jihlava (CZ)

(21) Appl. No.: 18/734,748

(22) Filed: Jun. 5, 2024

(51) Int. Cl. G16H 20/30; A61B 5/00; A61B 5/308

(52) U.S. Cl. CPC G16H 20/30; A61B 5/308; A61B 5/7267

(57) ABSTRACT

In a method of obtaining ventricular electrical activation parameters from an electrocardiogram signal, the electrocardiogram signal is pre-processed to remove baseline wandering to normalize the signal and optionally to amplify oscillations. The pre-processed electrocardiogram signal is fed to a neural network trained to estimate ventricular electrical activation parameters from electrocardiogram signal pre-processed in the same manner. The e ventricular electrical activation parameters are obtained as an output from the trained neural network. A method of training a neural network is also provided.

### FIELD

The disclosed embodiments relate to methods of obtaining ventricular electrical activation parameters from an electrocardiogram signal (ECG).

### BACKGROUND

Assessing ventricular electrical activation patterns is a critical aspect of cardiac diagnostics, aiming to evaluate the pathways of electrical activity within the heart's ventricles. This assessment provides insights into the coordination and synchronization of ventricular contraction, which is essential for efficient cardiac function. The ultra-high-frequency ECG technique can describe these patterns numerically; however, it requires high sampling frequencies (typically 3-7 kHz or rather 4-6 kHz) and longer recording times (typically in minutes, at least 30 seconds, often 30-120 seconds). Until the present invention, the complex relationship to ECG has limited utilization of standard ECG (i.e., shorter recording times and lower sampling frequencies, typically recording time of 10 seconds, at 1 KHz frequency) in estimating numerical values of ventricular electrical activation properties. The ventricular electrical activation patterns may be described using parameters known from prior art, e.g. U.S. Pat. Nos. 11,517,243 and 9,949,655, which however require ultra-high-frequency ECG as a source of data to achieve sufficient accuracy for clinically relevant outcomes. The present invention aims at removing this problem.

### BRIEF DESCRIPTION OF THE DRAWINGS

FIG. 1 schematically shows the training method of the invention. A) building a dataset, B) training and evaluating a neural network.

FIG. 2 schematically shows the method of determining the ventricular electrical activation parameters based on the present invention.

### DETAILED DESCRIPTION

The present invention provides a method of obtaining ventricular electrical activation parameters from an electrocardiogram signal (ECG), comprising the steps of:
pre-processing the electrocardiogram signal to remove baseline wandering, to normalize the signal and optionally to amplify oscillations,
feeding the pre-processed electrocardiogram signal to a neural network trained to estimate ventricular electrical activation parameters from electrocardiogram signal pre-processed in the same manner, and
obtaining the ventricular electrical activation parameters as an output from the trained neural network.

The ventricular electrical activation parameters preferably include at least one of: Ventricular Electrical Dyssynchrony (VED), Ventricular Activation Duration (VDn), and Ventricular Activation Index (ACIn).

Obtaining ECG signal:

Electrocardiogram is a plurality of signals recorded by a plurality of measurement electrodes and presented as a plurality of signals in channels. The signals are measured in a frequency range above 0.2 Hz. Currently, the signals are typically measured in frequency ranges starting from 100 Hz and up to 1,000 Hz, but any measuring frequency range is compliant with the present invention. The electrocardiogram signal used in the method of obtaining ventricular electrical activation parameters is preferably a standard electrocardiogram signal obtained at sampling frequencies from 0.1 kHz to 1.1 kHz, most typically at sampling frequency about 1 kHz.

In electrocardiography, a “sensor” is an electrode attached to the surface of the human body. “Channel” refers to a digitized signal from the sensor. “Lead” means the resulting digitized signal assembled according to the standard montages used in cardiology. The signals are dependent on electrical potential (voltage) on time. Consecutive signal values (voltage) in a digitized signal are called “samples”.

The method processes an electrocardiogram comprising signals from at least two channels. Typically, 2 to 256 channels are used. 12-lead or 14-lead ECG is preferred. Signals recorded in channels of V1, V2, V3, V4, V5, V6 electrocardiography leads or V1, V2, V3, V4, V5, V6, V7 and V8 electrocardiography leads are particularly preferred. Signals from all channels, or signals only from some channels can be used in the method of the invention.

The step of measuring electrocardiogram signal, which precedes the signal pre-processing step, is carried out by an apparatus comprising at least two sensors measuring the electrocardiogram signal, wherein the output(s) of at least two sensors is connected to an input of one or more analogue amplifiers, and an output of the said one or more analogue amplifiers is connected to an input of one or more analogue signal to digital signal converters, and an output of the one or more analogue signal to digital signal converters is connected to a processing unit, wherein the at least two sensors, the one or more analogue amplifiers, and the one or more analogue signal to digital signal converters have the transmission bandwidth of at least 50 Hz.

In some embodiments, the apparatus contains the same number of sensors, analogue amplifiers and analogue signal to digital signal converters, i.e., for each sensor, there is an analogue amplifier and an analogue signal to digital signal converter.

Signal pre-processing:

The step of signal-preprocessing aims at removing wandering baseline, amplifying oscillations, and normalizing (more preferably, standardizing) signals. Baseline wandering removal means removal of very low frequency components (<1 Hz), which are not useful in computation of ventricular electrical activation parameters. Amplifying oscillations improves neural network ability to effectively use these oscillations in the required task. Finally, normalizing signals (usually via standardization) increases performance of neural networks and other machine learning approaches.

In one embodiment of the pre-processing step, signal from each ECG lead is pre-processed by subtracting consecutive samples, meaning that resultant pre-processed signal consists of differences between consecutive samples of the original signal. Resultant pre-processed signal is shorter than input ECG lead just by one sample.

In another embodiment of the pre-processing step, amplitude envelopes are generated in a single or multiple frequency ranges (preferably within the frequency range 20-400 Hz).

An envelope is a smooth curve outlining the extremes of the oscillating signal. In this invention, an upper envelope is considered as the envelope, i.e., the curve outlining the upper extremes of the signal.

The envelope may be an amplitude envelope or a power envelope. The amplitude envelope is an envelope outlining the amplitude extremes of the signal. The power envelope is an envelope outlining the power extremes of the signal (power=amplitude squared).

In preferred embodiments, the amplitude or power envelopes of the ECG lead are calculated using Hilbert transformation, or the amplitude envelopes of the ECG lead are calculated by filtration, conversion of the signal obtained in this way into an absolute value and smoothing it, or the power envelopes of the ECG lead are calculated by filtration, raising the ECG signal to the power of two and smoothing it.

Yet another embodiment of the pre-processing step involves normalization of ECG signals. The ECG signals are normalized, or, more specifically, standardized.

In one embodiment of normalization, standardized signal is computed for all samples in a signal from each lead, such that a signal mean is calculated from all samples in a signal from a lead, the signal mean is subtracted from each sample, and the result is divided by standard deviation for the lead signal.

In another embodiment of normalization, simple normalization of the signal from each lead to a scale between 0 and 1 or between −1 and 1 is calculated. In case of normalization to a scale between 0 and 1, for each sample from each lead the lead signal minimum is subtracted, and the result is divided by the lead signal variation range.

Normalization generally means bringing a plurality of signals to the same range or to a predefined range. Standardization is a specific type of normalization. Methods to normalize or standardize signal are known to a person skilled in the art of signal processing.

The pre-processing step embodiments described herein may be used separately or may be combined. In particular, the normalization (preferably standardization) pre-processing step is preferably used, optionally in combination with other pre-processing steps.

The ventricular electrical activation parameters obtained from the method of the invention describe the ventricular activation pattern. The information relating to the ventricular activation pattern, i.e., the ventricular electrical activation parameters, can have various further use. For example, they can be used for selecting further treatment, wherein e.g. left-bundle-branch block patients with low VED do not benefit from cardiac resynchronization therapy. Alternatively, the determined ventricular electrical activation parameters can be used for optimization of pacemaker settings and/or pacing location wherein a clinician can determine, based on the ventricular electrical activation parameters, which deployment scenario leads to the most appropriate activation pattern.

The invention further provides a method of training a neural network suitable for use in the above-described method, comprising the steps of:
obtaining an ultra-high-frequency electrocardiogram signal recorded at sampling frequency above 1.1 kHz (preferably at sampling frequency above 2 kHz, or at sampling frequency above 3 kHz, or at sampling frequency above 4 kHz, or at sampling frequency 2 to 7 kHz, or at sampling frequency 3 to 7 kHz, or at sampling frequency 4 to 7 kHz),
obtaining the values of ventricular electrical activation parameter(s) from the ultra-high-frequency electrocardiogram signal,
down-sampling the ultra-high-frequency electrocardiogram signal to sampling frequency lower than or equal to 1.1 kHz to obtain a standard-frequency electrocardiogram signal,
pre-processing the standard-frequency electrocardiogram signal to remove wandering baseline, to normalize the signal and optionally to amplify oscillations,
using more than 50% of the pairs of the standard-frequency electrocardiogram signal and the obtained values of ventricular electrical activation parameter(s) as training data to train the neural network,
dividing the rest of the pairs of the standard-frequency electrocardiogram signal and the obtained values of ventricular electrical activation parameter(s) into validation data for training validation and as test data for final evaluation of the neural network.

The ultra-high-frequency electrocardiogram signal is an electrocardiogram signal obtained at sampling frequency above 1.1 kHz, preferably at sampling frequency above 2 kHz, or at sampling frequency above 3 kHz, or at sampling frequency above 4 kHz, or at sampling frequency 2 to 7 kHz, or at sampling frequency 3 to 7 kHz, or at sampling frequency 4 to 7 kHz.

The ultra-high-frequency electrocardiogram comprises signals from at least two channels. Typically, 2 to 256 channels are used. 12-lead or 14-lead ECG is preferred. Signals recorded in channels of V1, V2, V3, V4, V5, V6 electrocardiogram V1, V2, V3, V4, V5, V6, V7 and V8 electrocardiography leads are particularly preferred. Signals from all channels, or signals only from some channels can be used in the method of the invention.

The step of measuring electrocardiogram signal, which precedes the signal pre-processing step, is carried out by an apparatus comprising at least two sensors measuring the electrocardiogram signal, wherein the output(s) of at least two sensors is connected to an input of one or more analogue amplifiers, and an output of the said one or more analogue amplifiers is connected to an input of one or more analogue signal to digital signal converters, and an output of the one or more analogue signal to digital signal converters is connected to a processing unit, wherein the at least two sensors, the one or more analogue amplifiers, and the one or more analogue signal to digital signal converters have the transmission bandwidth of at least 0.3 kHz.

In some embodiments, the apparatus contains the same number of sensors, analogue amplifiers and analogue signal to digital signal converters, i.e., for each sensor, there is an analogue amplifier and an analogue signal to digital signal converter.

Methods of obtaining the values of ventricular electrical activation parameters from UHF-ECG are known in the art, e.g. in U.S. Pat. No. 11,517,243 or in U.S. Pat. No. 9,949,655.

For example, a method of obtaining the values of VDn from ultra-high-frequency ECG may comprise the following steps:
providing an electrocardiogram at a sampling frequency above 1.1 kHz, preferably at sampling frequency above 2 kHz, or at sampling frequency above 3 kHz, or at sampling frequency above 4 kHz, or at sampling frequency 2 to 7 kHz, or at sampling frequency 3 to 7 kHz, or at sampling frequency 4 to 7 kHz, said electrocardiogram comprising signals in at least two channels;
selecting at least two frequency ranges of the signal in each of the said at least two channels;
calculating an envelope for the signal in each frequency range in each channel;
dividing the calculated envelope of the signal in each frequency range in each channel into QRS complex segment envelopes;
computing an average or median envelope as an average or mean of all QRS complex segment envelopes for each frequency range in each channel;
optionally performing baseline correction for each average or median envelope by subtracting mean or median value from an interval in which QRS complex is not present (i.e., interval between QRS complexes) to remove noise background,
normalizing the average or median envelope to obtain a normalized average or median envelope for each frequency range in each channel; wherein the normalization is performed by dividing the average or median envelope of each frequency range in each channel by its integral or by a maximal value reached in the average or median envelope, in each frequency range and each channel separately, wherein the integral or the maximal value are calculated within an interval of a minimum of 50 ms before the position of QRS complex and minimum 50 ms after the position of QRS complex;
calculating a signal average or median envelope from the normalized average or median envelopes of all frequency ranges within each channel; and
obtaining the values of Ventricular Activation Duration VDn of the signal average or median envelope as time length of a horizontal line crossing the signal average or median envelope, wherein the horizontal line is at a level corresponding to a pre-determined value within the range of 10-70 percent of the maximum value of the signal average or median envelope.

For example, a method of obtaining the values of ACIn may correspond to the method of computing VDn, but may further include a step of calculating a volumetric activation index ACIn as an area delimited by the signal average or median envelope and horizontal line, wherein the horizontal line is at a level corresponding to a pre-determined value within the range of 10-70 percent of the maximum value of the signal average or median envelope or a final average or median envelope, wherein the value of the signal average or median envelope or a final average or median envelope is normalized to 0 at the threshold level and 1 at the maximum level. This step may, for example, follow the step of calculating the VDn.

For example, a method of obtaining the value of VED may comprise a step of calculating an activation time (ATi) as time position of the center of mass of signal normalized average or median envelopes above the horizontal line crossing signal normalized average or median envelopes at a predetermined level which is pre-set within a range of 10-70 percent of the maximum of signal normalized average or median envelopes or time position of maximal value of signal normalized average or median envelopes, and subsequently calculating ventricular electrical dyssynchrony-VED—as time difference between activation times of two or more ECG leads. The VED parameter indicates a time delay of ventricular depolarization between any two or more ECG leads. The relevant value is the highest value achieved for any combination of two or more leads used in the method of the invention.

Preferred procedure for preparing averaged envelopes:

At least two non-overlapping frequency ranges are selected in each of the said at least two leads. The frequency ranges are frequency bands above the frequency of 0.2 Hz. The width of each frequency range may preferably be from 20 to 1,000 Hz. The frequency ranges are preferably the same in each channel.

An envelope of the signal is calculated for each frequency range in each lead. An envelope is a smooth curve outlining the extremes of the oscillating signal. In this invention, an upper envelope is considered as the envelope, i.e., the curve outlining the upper extremes of the signal.

The envelope may be an amplitude envelope or a power envelope. The amplitude envelope is an envelope outlining the amplitude extremes of the signal. The power envelope is an envelope outlining the power extremes of the signal (power=amplitude squared).

In preferred embodiments, the amplitude or power envelopes of the ECG lead are calculated using Hilbert transformation, or the amplitude envelopes of the ECG lead are calculated by filtration, conversion of the signal obtained in this way into an absolute value and smoothing it, or the power envelopes of the ECG lead are calculated by filtration, raising the ECG signal to the power of two and smoothing it.

The calculated envelope of the signal in each frequency range in each lead is divided into QRS complex envelopes, wherein a QRS complex envelope is a portion of the envelope of the signal, said portion corresponding to one QRS complex, i.e., outlining one QRS complex. A QRS complex is the combination of three of the graphical deflections shown on an electrocardiogram, wherein the QRS complex corresponds to the depolarization of the right and left ventricles. QRS complex contains the waves Q, R and S. Q and S waves are downward deflections and R is an upward deflection The position of the QRS complex (also called “QRS complex annotation”) is detected and annotated by known algorithms such as Pan-Tompkins or Hilbert transform algorithms. Many other algorithms are available and known to a person skilled in the art. The annotation algorithms annotate all QRS complexes in one frequency range in the same way and all QRS complexes in all frequency ranges and in all leads in the same way.

QRS complex envelope is preferably a portion of the envelope of the signal which starts at least 50 ms, or 50 to 500 ms, or 50 to 150 ms, or 120 to 200 ms before the annotation of the QRS complex, and ends at least 50 ms, or 50 to 500 ms, or 50 to 150 ms, or 120 to 200 ms after the annotation of the QRS complex.

An average envelope or a median envelope is then computed from the QRS complex envelopes within each of the frequency ranges, in each of the leads. This step increases a signal-to-noise ratio for each frequency range in each lead.

Baseline correction may optionally be performed for each average envelope or median envelope by subtracting the mean (average) or median value from a temporal interval in which no QRS complex is present, in order to remove noise background. Baseline correction is particularly useful if the integral is used in the following step of normalization. The interval in which no QRS complex is present is an interval anywhere between the S wave of one QRS complex and the Q wave of the following QRS complex.

The average envelope or median envelope are normalized to obtain a normalized average envelope or normalized median envelope for each frequency range in each lead. The normalization is performed by dividing the average envelope or the median envelope of each frequency range in each channel by its integral or by a maximal value reached in the average envelope or median envelope. The integral or the maximal value is calculated within an interval of a minimum of 50 ms before the QRS complex annotation and a minimum of 50 ms after the QRS complex annotation. One normalized average or median envelope is obtained per each frequency range, in each lead.

Calculations of average, median, or normalization are performed in the sequence of points whose time distance from the QRS complex annotation is equal. In other words, each point (e.g., sampling point) of the average, median, or normalized envelope is calculated as an average, median, or normalized value, respectively, of the points in the same temporal position of all envelopes over which the calculation of the average, median or normalization is performed.

In some preferred embodiments, the method may further comprise the step of calculating a final average or median envelope from all signal average or median envelopes of the said at least two leads.

Preferred procedure for calculation of VED, ACIn and VDn features from UHF-ECG:

The Ventricular Activation Duration—VDn—is the time length of a horizontal line crossing the signal average or median envelope, wherein the horizontal line is at a predetermined level which is pre-set within a range of 10-70 percent of the maximum value of the signal average or median envelope. The “n” refers to the index of the ECG lead. This feature was introduced in U.S. Pat. No. 11,517,243 (under the designation Vdi, where “i” refers to the index of the ECG lead).

A Ventricular activation index—ACIn—is an area delimited by the signal average or median envelope and horizontal line, wherein the horizontal line is at a predetermined level which is pre-set within a range of 10-70 percent of the maximum value of the signal average or median envelope or a final average or median envelope, wherein the value of the signal average or median envelope or a final average or median envelope is normalized to 0 at the threshold level and 1 at the maximum level. The “n” refers to the index of ECG lead. ACIn provides information corresponding to the number of simultaneously activated myocardial cells in a local volume. ACIn was introduced in U.S. Pat. No. 11,517,243 (under a “Ali” where “i” refers to index of ECG lead)

In one preferred embodiment of the invention, the method further comprises a step of calculating an activation time (ATi) as time position of the center of mass of signal normalized average or median envelopes above the horizontal line crossing signal normalized average or median envelopes at a predetermined level which is pre-set within a range of 10-70 percent of the maximum of signal normalized average or median envelopes or time position of maximal value of signal normalized average or median envelopes, and subsequently calculating ventricular electrical dyssynchrony—VED—as time difference between activation times of two or more ECG leads. The VED parameter indicates a time delay of ventricular depolarization between any two or more ECG leads. The relevant value is the highest value achieved for any combination of two or more leads used in the method of the invention. VED computation was described in the patent U.S. Pat. No. 9,949,655, where it is called “distance D.”

ECG signals are down-sampled using common approaches to down-sampling signals, generally known to those skilled in the art (e.g., from Orhan Gazi: Understanding Digital Signal Processing, Springer Nature, ISBN 9789811352775; or from Lathi B. P., Green R. A.: Essentials of Digital Signal Processing, ISBN-10 1107059321). Down-sampling refers to decreasing the sampling frequency of the ECG signal. In this invention, down-sampling refers in particular of decreasing the sampling frequency of the ECG signal from above 1.1 kHz, preferably from above 4 kHz, (UHF-ECG) to standard ECG sampling frequency of below 1.1 kHz (typically about 1 kHz).

The step of pre-processing of the down-sampled ECG signal may be carried out as described herein above. It is strongly preferred that the pre-processing of the down-sampled ECG signal used for training of the neural network is the same as the pre-processing which would then be used for the signals fed to the trained neural network.

The training, validation and test data are pairs: the standard-frequency electrocardiogram signal (obtained by down-sampling of the UHF-ECG signal) and the corresponding values of ventricular electrical activation parameter(s) (obtained by calculating from the UHF-ECG signal).

In principle, any type of neural network can be used in the methods of the invention. Preferably, convolutional neural network is used.

Neural network input is defined by the number and size of pre-processed ECG lead signals; the output vector contains each of ventricular electrical activation parameters which are obtained through the trained neural network. Preferably, the neural network contains convolutional layers (accompanied by pooling layers) since these layers can be trained to recognize morphological features from the signal invariantly on their absolute location in a signal. The neural network preferably contains drop-out layers to improve generalization ability of the network. The neural network preferably contains at least one fully connected layer before the output layer.

In some embodiments, the neural network may be based on a model comprising a plurality (preferably 3 to 11, more preferably 5 to 7) identical blocks with different sets of hyperparameters. Each block contains a twice repeated sequence of layers: a convolutional layer, an instance normalization layer, and ReLU (Rectified Linear Unit) activation function. Each of these blocks is followed by a MaxPooling layer that shrinks the input length into half and drop-out layer. Finally, a MaxPooling layer and two fully connected layers close the architecture with the output neuron count corresponding to the number of the ventricular electrical activation parameters to be obtained. The training comprises iterative optimization of network weights. After each “epoch” (block of iterations when all training data were shown to the network), the error is evaluated on validation and training set.

In some embodiments, a process called transfer-learning can be used in the step of training the neural network. Instead of training the neural network from a scratch, already existing and trained neural network (with compatible inputs) is used and only the last 1 or 2 layers are modified and fine-tuned for the present purpose using the procedure and parameters described in the previous embodiment. This means that a significantly lower amount of data (hundreds, instead of thousands) is required for proper neural network training.

In some embodiments, output states from hidden fully connected layer can be used as feature sets, supplied to common machine learning mechanisms such as supported vector machines, random forests etc. The last layer is trained using the procedure described in the one-before-previous embodiment. This means that a significantly lower amount of data (hundreds) is required for proper neural network training.

In all the above-described embodiments of the training step, the training is preferably stopped when the model error on validation dataset stops decreasing.

The present invention further provides an apparatus for processing electrocardiographic signal. Said apparatus comprises:
one or more analogue amplifiers each including an input and an output, the input of each of the analogue amplifiers being connected to an output of a sensor of the ECG signal,
one or more analogue signal to digital signal converters each including an input and an output, the input of each of the analogue signal to digital signal converters being connected to the output of a corresponding one of the one or more analogue amplifiers, wherein the sensors, the analogue amplifiers, and the analogue signal to digital signal converters have the transmission bandwidth at least 50 Hz, and
a processing unit including an input connected to the output of the analogue to digital signal converters and an output configured to be connected to at least one displaying unit, wherein the processing unit comprises a trained neural network configured to carry out the method of the present invention. The trained neural network is trained by the method of training according to the present invention.

The displaying unit is configured to display ventricular electrical activation parameters.

### EXAMPLE

This chapter describes an example of the novel approach to estimate ventricular electrical activation parameters from standard ECG signals, using a convolutional neural network (CNN), including the training of the CNN. The example comprises an approach to create dataset, a pre-processing step for signal transformation, a CNN model training on an annotated ECG dataset, and estimation of ventricular electrical activation parameters based on the CNN output.

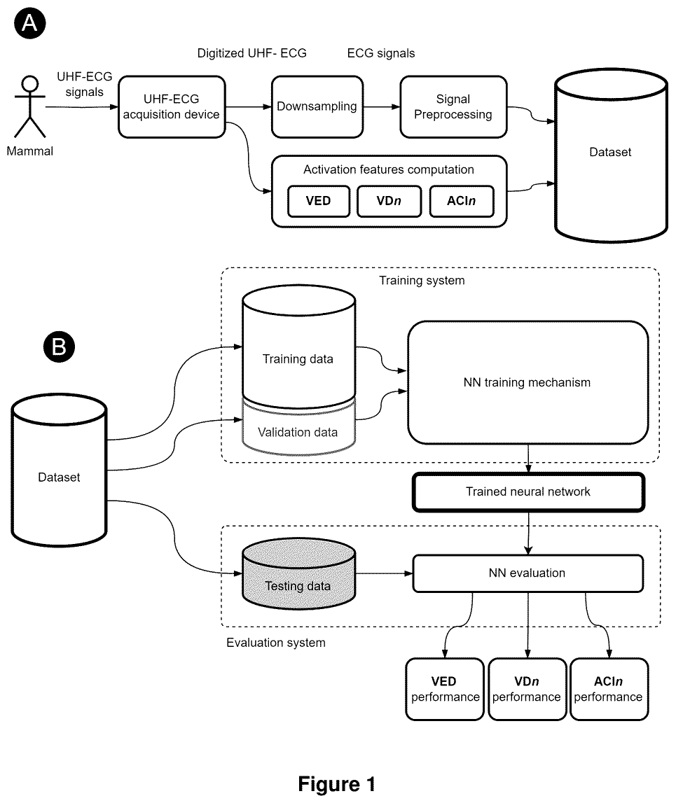

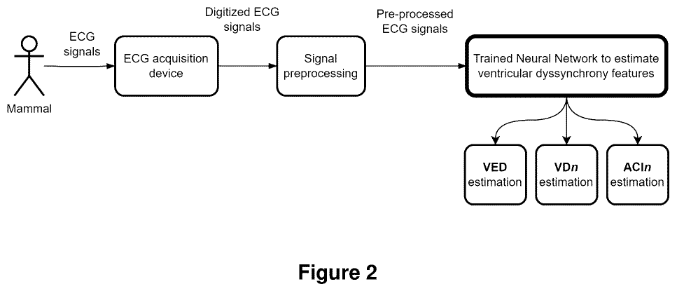

FIGS. 1 and 2 show schematically the workflows of the training (FIG. 1) and the use of the trained neural network (FIG. 2).

FIG. 1A shows the steps of Building a Dataset. FIG. 1B shows the neural network training. UHF-ECG signals from patients at resting supine position were recorded at a sampling rate of 5 kHz, at a length of 239.7 L 160.2 seconds (FIG. 1A). Output is a data block containing ECG leads.

For each UHF-ECG recording, target values (N=13) of ventricular activation features were determined using the software VDI Scientific (VDI Technologies, Czechia). Single VED value was determined using multiple ECG leads; furthermore, VDn, and ACIn were computed for each ECG lead n using the same software tool. The software VDI Scientific is based on the methods for calculation of VED, VDn and ACIn from UHF-ECG spectra described herein above.

Signals from UHF-ECG leads from each recording (N=7,552) were re-sampled using the zero phase low-pass FIR filter, further down-sampled with factor 5 to 1 kHz sampling frequency. Signals were trimmed to the 10 seconds length.

ECG signals were filtered to remove baseline wandering and to amplify oscillations using value differences between consecutive samples in the signal.

In an alternative method of pre-processing, amplitude envelopes could be generated in a single or multiple frequency ranges.

Scale of filtered ECG leads was changed—it was standardized as follows: from each sample in a signal, the signal mean is subtracted, and the result is divided by signal standard deviation. In an alternative, scale may be normalized between <0, 1> or <−1, 1>.

Down-sampled and re-scaled ECG lead signals and corresponding ventricular electrical activation parameters (as calculated from the original UHF-ECG signals) formed a dataset.

This dataset was split into the training, validation and test subsets at quantities of 5,800, 568, and 1,184, respectively (FIG. 1B). Training subset was used for training of the model weights, validation subset was used to check against overfitting and testing subset for reporting results. We defined model architecture for preferable inputs: The architecture comprises 5 identical blocks with different sets of hyperparameters. Each block consists of a twice repeated sequence of layers: a convolutional layer, an instance normalization layer, and ReLU activation function. The kernel size in the first convolutional layer is set to 5 and in the second convolutional layer set to 7. The depth of the output feature map from these 5 blocks is 20, 40, 80, 160, and 320, respectively. Each of these blocks is followed by a MaxPooling layer that shrinks the input length into half and drop-out layer with probability of 0.5. Finally, a MaxPooling layer and two fully connected layers closes the architecture with neuron counts 160 and 13, respectively.

Training comprises iterative optimization of network weights. After each “epoch” (block of iterations when all training data were shown to the network), the error on validation and training set was evaluated.

Alternatively, a process called transfer-learning can be used. This means that instead of training NN from a scratch, an already existing and trained NN (with compatible inputs) is used and only the last 1-2 layers are modified and fine-tuned for the present purpose. This means that a significantly lower amount of data (hundreds) is required for proper NN training. Or, output states from hidden fully connected layer can be used as feature sets, supplied to common machine learning mechanisms such as supported vector machines, random forests etc.

Training was stopped when the model error on validation dataset stopped decreasing (in the preferred solution, this happened after 16 epochs). Neural network was saved for later inference.

Evaluation on test dataset showed acceptable mean absolute error (in milliseconds):

| Feature | VED | VD1 | VD2 | VD3 | VD4 | VD5 | VD6 | ACI1 | ACI2 | ACI3 | ACI4 | ACI5 | ACI6 |
|---|---|---|---|---|---|---|---|---|---|---|---|---|---|
| Mean | 13.7 | 11.0 | 9.6 | 9.1 | 9.4 | 8.9 | 10.0 | 8.5 | 7.8 | 7.4 | 7.6 | 7.3 | 7.9 |
| abs. | | | | | | | | | | | | | |
| Error | | | | | | | | | | | | | |

The trained neural network was used for determining ventricular electrical activation parameters as follows. FIG. 2 shows the method schematically.

Standard ECG signals from patients at resting supine position were recorded at a sampling rate of 0.8 kHz (more generally, sampling rates of up to and including 1.0 kHz are typically used), at a length of at least 6 seconds, preferably 10 seconds or longer.

Signals were resampled to the same frequency which was used during NN training (preferably 1 kHz). Signals were trimmed to the length required by NN.

Baseline wandering was removed, higher frequencies were emphasized in the same way as used during NN training. Signals were normalized in the same way as used during NN training.

The thus pre-processed signals were fed into the trained neural network. After inference, the network outputs at least 3 (preferably 13) continuous features: VED, VDn, and ACIn (where n refers to the index of ECG lead).

### INDUSTRIAL APPLICABILITY

The invention has the following advantages:

Cost-effectiveness: The invention allows to utilize standard ECG equipment (typically 1 kHz sampling frequency), making it accessible in various healthcare settings.

Time-efficiency: Offers shorter measurement times compared to UHF-ECG.

Fast pre-processing: While the original UHF-ECG approach requires complex detection and signal preparation, the use of the present invention requires only removal of baseline wandering (differentiation) and standardization.

Scalability: The CNN model can be used in a web service or directly on-site as a part of an ECG device.

The methods described herein have broad applications in both pre-clinical and clinical cardiology.

They can assist in the early detection and management of cardiovascular diseases by providing a timely and accurate assessment of parameters describing ventricular electrical activation. Examples are pre-surgical decision support before pacemaker implantation, monitoring during pacemaker implantation, and support in pacemaker fine-tuning.

What is claimed is:

1. A method of obtaining ventricular electrical activation parameters from an electrocardiogram signal, said method comprising the steps of:
pre-processing the electrocardiogram signal to remove baseline wandering, to normalize the signal, and optionally to amplify oscillations,
feeding the pre-processed electrocardiogram signal to a neural network trained to estimate ventricular electrical activation parameters from electrocardiogram signals pre-processed in the same manner, and
obtaining the ventricular electrical activation parameters as an output from the trained neural network.

2. The method of claim 1, wherein the ventricular electrical activation parameters include at least one of: Ventricular Electrical Dyssynchrony (VED), Ventricular Activation Duration (VDn), and Ventricular Activation Index (ACIn).

3. The method of claim 1, wherein the electrocardiogram signal has a sampling frequency from 0.1 kHz to 1.1 kHz.

4. The method of claim 1, wherein the step of pre-processing the electrocardiogram signal includes subtracting consecutive samples, wherein the resultant pre-processed signal consists of differences between consecutive samples of the original signal.

5. The method of claim 1, wherein the step of pre-processing the electrocardiogram signal includes computing a standardized signal which is computed for all samples in a signal from each lead, such that a signal mean is calculated from all samples in a signal from a lead, the signal mean is subtracted from each sample, and the result is divided by standard deviation for the lead signal, and/or the step of pre-processing the electrocardiogram signal includes normalization of the signal from each lead to a scale between 0 and 1 or between −1 and 1.

6. A method of training a neural network, said method of training comprising the steps of:
obtaining a plurality of ultra-high-frequency electrocardiogram signals recorded at sampling frequencies above 1.1 kHz,
obtaining values of ventricular electrical activation parameter(s) from each ultra-high-frequency electrocardiogram signal,
down-sampling each ultra-high-frequency electrocardiogram signal to a sampling frequency lower than or equal to 1.1 kHz to obtain a standard-frequency electrocardiogram signal,
pre-processing each standard-frequency electrocardiogram signal to remove wandering baseline, to normalize the signal, and optionally to amplify oscillations,
using more than 50% of all pairs of the plurality of standard-frequency electrocardiogram signals and the associated obtained values of ventricular electrical activation parameter(s) as training data to train the neural network, and
dividing the rest of the pairs of the plurality of standard-frequency electrocardiogram signals and the associated obtained values of ventricular electrical activation parameter(s) into validation data for training validation and as test data for final evaluation of the neural network.

7. The method according to claim 6, wherein the step of pre-processing the standard-frequency electrocardiogram signal includes subtracting consecutive samples, wherein the resultant pre-processed signal consists of differences between consecutive samples of the original signal.

8. The method according to claim 6, wherein the step of pre-processing the standard-frequency electrocardiogram signal includes computing a standardized signal which is computed for all samples in a signal from each lead, such that a signal mean is calculated from all samples in a signal from a lead, the signal mean is subtracted from each sample, and the result is divided by standard deviation for the lead signal, and/or the step of pre-processing the electrocardiogram signal includes normalization of the signal from each lead to a scale between 0 and 1 or between −1 and 1.

9. The method according to claim 6, wherein the step of pre-processing the standard-frequency electrocardiogram signal for training of the neural network is the same as the pre-processing for the signals fed to the trained neural network.

10. An apparatus for processing electrocardiographic, said apparatus comprising:
one or more analogue amplifiers each including an input and an output, the input of each of the analogue amplifiers being connected to an output of a sensor of the ECG signal,
one or more analogue signal to digital signal converters each including an input and an output, the input of each of the analogue signal to digital signal converters being connected to the output of a corresponding one of the one or more analogue amplifiers, wherein the sensors, the analogue amplifiers, and the analogue signal to digital signal converters have the transmission bandwidth at least 50 Hz, and
a processing unit including an input connected to the output of the analogue to digital signal converters and an output configured to be connected to at least one displaying unit, wherein the processing unit comprises a processor and a trained neural network which are configured to carry out the steps of:
pre-processing the electrocardiogram signal to remove baseline wandering, to normalize the signal, and optionally to amplify oscillations,
feeding the pre-processed electrocardiogram signal to a neural network trained to estimate ventricular electrical activation parameters from electrocardiogram signal pre-processed in the same manner, and
obtaining the ventricular electrical activation parameters as an output from the trained neural network.
